(19) United States
(12) Patent Application Publication
(10) Pub. No.: US 2021/0017671 A1
(43) Pub. Date: Jan. 21, 2021

(54) SPINNING MICROFIBRILLATED CELLULOSE

(71) Applicant: Stora Enso OYJ, Helsinki (FI)

(72) Inventors: Gisela Cunha, Nacka (SE); Heidi Saxell, Stockholm (SE)

(73) Assignee: Stora Enso OYJ, Helsinki (FI)

(21) Appl. No.: 16/955,910

(22) PCT Filed: Dec. 20, 2018
(86) PCT No.: PCT/IB2018/060413

§ 371 Date: Jun. 19, 2020

(30) Foreign Application Priority Data
Dec. 21, 2017 (SE) 1751615-4

(51) Int. Cl. D01F 2/30; D01D 5/00; D01D 5/04; D04H 3/015; D01D 5/06; C08B 5/00; D03D 15/00; A61L 15/28

(52) U.S. Cl. CPC D01F 2/30; D01D 5/0007; D01D 5/04; A61L 15/28; D01D 5/06; C08B 5/00; D03D 15/00; D04H 3/015

(57) ABSTRACT

A method is provided for preparing a fibrous material (preferably a mat or filaments) of crosslinked microfibrillated cellulose. Phosphorylated microfibrillated cellulose is spun into a fibrous material; and then said fibrous material is post-treated (e.g. by heat-treatment) to provide crosslinking between the phosphorylated microfibrillated cellulose. Fibrous materials such as filaments or mats, and hygiene products comprising such materials are also described.

A method for providing a fibrous material of crosslinked microfibrillated cellulose is provided, as well as a spun fibrous material of crosslinked phosphorylated microfibrillated cellulose. Products comprising said fibrous material are also described. Such fibrous materials exhibit desirable properties, e.g. strength (in particular wet-strength), water absorbance and elasticity/flexibility.

### BACKGROUND

Microfibrillated cellulose (MFC) comprises partly or totally fibrillated cellulose or lignocellulose fibers. The liberated fibrils have a diameter less than 100 nm, whereas the actual fibril diameter or particle size distribution and/or aspect ratio (length/width) depends on the source and the manufacturing methods. The smallest fibril is called elementary fibril and has a diameter of approximately 2-4 nm (see e.g. Chinga-Carrasco, G., Nanoscale research letters 2011, 6:417), while it is common that the aggregated form of the elementary fibrils, also defined as microfibril, is the main product that is obtained when making MFC e.g. by using an extended refining process or pressure-drop disintegration process (see Fengel, D., Tappi J., March 1970, Vol 53, No. 3.). Depending on the source and the manufacturing process, the length of the fibrils can vary from around 1 to more than 10 micrometers. A coarse MFC grade might contain a substantial fraction of fibrillated fibers, i.e. protruding fibrils from the tracheid (cellulose fiber), and with a certain amount of fibrils liberated from the tracheid (cellulose fiber).

There are different acronyms for MFC such as cellulose microfibrils, fibrillated cellulose, nanofibrillated cellulose, fibril aggregates, nanoscale cellulose fibrils, cellulose nanofibers, cellulose nanofibrils, cellulose microfibers, cellulose fibrils, microfibrillar cellulose, microfibril aggregates and cellulose microfibril aggregates. MFC can also be characterized by various physical or physical-chemical properties such as large surface area or its ability to form a gel-like material at low solids (1-5 wt %) when dispersed in water.

MFC exhibits useful chemical and mechanical properties. Chemical surface modification of MFC has the potential to improve the properties of MFC itself, as well as filaments spun from MFC, e.g. mechanical strength, water absorbance and elasticity/flexibility.

In a recent review article, Lundahl et al. Ind. Eng. Chem. Res., 2017, 56 (1), pp 8-19 provide an overview of methods for spinning MFC into filaments. Among other things, filaments obtained from spinning TEMPO-oxidised MFC are shown to be weaker than filaments spun from non-treated MFC.

An additional problem with chemically modified MFC is that it has increased water absorption when compared to non-modified MFC, due to its chemical charge, and can start losing integrity upon contact with water. A balance of mechanical strength and water absorbance can therefore be difficult to achieve.

Other documents in this technical field include U.S. Pat. Nos. 4,256,111 and 6,027,536.

There is therefore a need to improve the properties of mats or filaments spun from MFC; in particular, (wet) strength, water absorption and elasticity/flexibility properties. Suitably, the improvement can be achieved in a straightforward manner, without the use of external modifiers such as crosslinkers.

### SUMMARY

It has been found by the present inventor(s) that fibrous materials (e.g. a mat or filaments) with desirable elasticity and water absorption can be formed from a cellulose composition comprising phosphorylated microfibrillated cellulose (P-MFC).

A method for preparing a fibrous material (e.g. filaments or amat) of crosslinked microfibrillated cellulose is thus provided, said method comprising the steps of:
i. forming a cellulose composition comprising or consisting of phosphorylated microfibrillated cellulose (P-MFC) into a fibrous material;
ii. heat-treatment of said fibrous material to provide crosslinking of the phosphorylated microfibrillated cellulose.

A spun fibrous material obtained via the method described herein is also provided, said fibrous material being e.g. a spun mat or spun filaments. Additionally, spun fibrous material of crosslinked phosphorylated microfibrillated cellulose, being a spun mat or spun filaments is provided. A web containing such spun filaments is also provided, as is a water-absorbent material comprising the spun fibrous material. In a further aspect, a hygiene product comprising the spun fibrous material and/or water-absorbent material is provided.

Further aspects of the invention are provided in the following text and in the dependent claims.

### DETAILED DISCLOSURE

In a first aspect, the invention provides a method for preparing a fibrous material of crosslinked microfibrillated cellulose (MFC). The term “fibrous material” is used herein includes mats and filaments, preferably filaments.

Microfibrillated cellulose (MFC) or so called cellulose microfibrils (CMF) shall in the context of the patent application mean a nano-scale cellulose particle fiber or fibril with at least one dimension less than 100 nm. MFC comprises partly or totally fibrillated cellulose or lignocellulose fibers. The cellulose fiber is preferably fibrillated to such an extent that the final specific surface area of the formed MFC is from about 1 to about 300 m²/g, such as from 1 to 200 m²/g or more preferably 50-200 m²/g when determined for a freeze-dried material with the BET method.

Various methods exist to make MFC, such as single or multiple pass refining, pre-hydrolysis followed by refining or high shear disintegration or liberation of fibrils. One or several pre-treatment steps are usually required in order to make MFC manufacturing both energy efficient and sustainable. The cellulose fibers of the pulp to be supplied may thus be pre-treated enzymatically or chemically, for example to reduce the quantity of hemicellulose or lignin. The cellulose fibers may be chemically modified before fibrillation, wherein the cellulose molecules contain functional groups other (or more) than found in the original cellulose. Such groups include, among others, carboxymethyl (CMC), aldehyde and/or carboxyl groups (cellulose obtained by N-oxyl mediated oxidation, for example “TEMPO”), or quaternary ammonium (cationic cellulose). After being modified or oxidized in one of the above-described methods, it is easier to disintegrate the fibers into MFC or NFC.

The nanofibrillar cellulose may contain some hemicelluloses; the amount is dependent on the plant source. Mechanical disintegration of the pre-treated fibers, e.g. hydrolysed, pre-swelled, or oxidized cellulose raw material is carried out with suitable equipment such as a refiner, grinder, homogenizer, colloider, friction grinder, ultrasound sonicator, single- or twin-screw extruder, fluidizer such as microfluidizer, macrofluidizer or fluidizer-type homogenizer. Depending on the MFC manufacturing method, the product might also contain fines, or nanocrystalline cellulose or e.g. other chemicals present in wood fibers or in papermaking process. The product might also contain various amounts of micron size fiber particles that have not been efficiently fibrillated.

MFC can be produced from wood cellulose fibers, both from hardwood or softwood fibers. It can also be made from microbial sources, agricultural fibers such as wheat straw pulp, bamboo, bagasse, or other non-wood fiber sources. It is preferably made from pulp including pulp from virgin fiber, e.g. mechanical, chemical and/or thermomechanical pulps. It can also be made from broke or recycled paper.

The above described definition of MFC includes, but is not limited to, the proposed TAPPI standard W13021 on cellulose nano or microfibril (CMF) defining a cellulose nanofiber material containing multiple elementary fibrils with both crystalline and amorphous regions, having a high aspect ratio with width of 5-30 nm and aspect ratio usually greater than 50.

Phosphorylated microfibrillated cellulose (P-MFC) is typically obtained by reacting cellulose pulp fibers with a phosphorylating agent such as phosphoric acid, and subsequently fibrillating the fibers to P-MFC. One particular method involves providing a suspension of cellulose pulp fibers in water, and phosphorylating the cellulose pulp fibers in said water suspension with a phosphorylating agent, followed by fibrillation with methods common in the art. Suitable phosphorylating agents include phosphoric acid, phosphorus pentaoxide, phosphorus oxychloride, diammonium hydrogen phosphate and sodium dihydrogen phosphate.

In the reaction to form P-MFC, alcohol functionalities (—OH) in the cellulose are converted to phosphate groups (—OPO₃²⁻). In this manner, crosslinkable functional groups (phosphate groups) are introduced to the pulp fibers or microfibrillated cellulose.

In a first general step of the method, cellulose composition comprising or consisting of phosphorylated microfibrillated cellulose (P-MFC) is spun into a fibrous material.

In the case that the cellulose composition consists of P-MFC, no components other than P-MFC are present in the composition. In the case that the cellulose composition comprises P-MFC, components other than P-MFC may be present in the composition. However, the cellulose composition suitably comprises more than 25%, preferably more than 50%, such as e.g. more than 75% by weight P-MFC. In one preferred aspect, the cellulose composition comprising P-MFC may additionally comprise unmodified (native) MFC. By “unmodified” or “native” MFC is meant microfibrillated cellulose which is the direct result of fibrillation of native cellulose fibers, i.e. without chemical treatment before or after fibrillation. Suitably, therefore, the cellulose composition consists of P-MFC and MFC. Alternatively or additionally, the cellulose composition comprising P-MFC may additionally comprise chemically-modified microfibrillated cellulose, such as e.g. dialdehyde-MFC or TEMPO-MFC (i.e. MFC oxidised with 2,2,6,6-tetramethylpiperidin-1-yl)oxidanyl). Additional components of the cellulose composition may include natural or synthetic filaments or natural or synthetic staple fibres.

In a second general step of the method, the fibrous material from the first step is heat-treated so as to provide crosslinking of the phosphorylated microfibrillated cellulose. Crosslinking suitably takes place without the use of any additional crosslinking agents; i.e. crosslinks are formed directly between the phosphate moieties and other components of the cellulose composition.

Heat treatment in the second general step of the method suitably takes place at a temperature of between 60 and 200° C., e.g. between 70 and 120° C. Such temperatures are sufficient to obtain crosslinking, but also limit potential degradation of the MFC. It has been established that heat treatment suitably takes place for a time of between 10 and 180 minutes, depending on the temperature used and initial solids content of the material to heat treat. Heat treatment may take place in an oven, but other methods of heat treatment may also be used.

The fibrous material is preferably filaments, and the forming process is spinning. General methods for spinning filaments from MFC are described e.g. in Lundahl et al. Ind. Eng. Chem. Res., 2017, 56 (1), pp 8-19. Suitable spinning processes may be selected from wet-spinning, electrospinning and dry-spinning. A preferred spinning process for phosphorylated microfibrillated cellulose is dry-spinning, as this technique avoids the need for an additional coagulation bath and makes it easier to handle the filaments and create patterns (e.g. grids).

The fibrous material may also be a mat. If the fibrous material is a mat, the composition is spun. By “spun mat” is mean that—instead of spinning a single filament—one can directly spun an interconnected structure made of filaments.

The general steps of the method (spinning, followed by heat-treatment) may be carried out without any intervening method steps. Alternatively, one or more intervening method steps may be carried out between the spinning step and the heat-treatment step. In one particular aspect, the fibrous material may be dried before or during the heat-treatment step. Drying can suitably take place under ambient conditions (e.g. 25° C.). It has been discovered that crosslinking can be triggered in fibrous material which has been previously dried at ambient conditions, e.g. by putting dried fibrous material according to the invention in the oven. This means that one can in principle dry the material at ambient conditions (with no crosslinking) and then trigger the crosslinking when desired at a later stage by heat-treatment.

Alternatively, the step of drying the fibrous material (mat or filaments) can take place during the heat-treatment step. In this alternative, a dry, crosslinked fibrous material is obtained, which can have advantageous water-absorptive and strength properties both in dry and wet conditions.

If hydrated fibrous material is required, a further step of hydrating said fibrous material with water after the heat-treatment step may be carried out.

It is thought not to be enough to remove the water from the sample at room temperature (i.e. to dry at RT); a heat-treatment is required for the crosslinking. Furthermore, it was considered surprising that some stretchability/elasticity behaviour could be obtained after soaking the heat-treated material in water.

The general method of the invention can be used to provide spun filaments of crosslinked phosphorylated microfibrillated cellulose. The spun filaments can—in turn—be used to prepare a web of spun filaments, by laying said spun filaments to provide a web. The invention therefore provides a web comprising spun filaments, wherein said spun filaments are as described herein.

The web may comprise additional filaments or fibres such as e.g. synthetic filaments, wood fibres or spun filaments of non-modified MFC or other types of modified MFC. The web may be woven or non-woven. The web may be an air-laid, melt-blown or spunlaid non-woven web.

The present invention also provides a spun mat or spun filaments, preferably spun filaments, obtained via the method described herein. Additionally provided is a spun mat or spun filaments of crosslinked phosphorylated microfibrillated cellulose. The presence of phosphate crosslinks between MFC fibrils can be ascertained by spectroscopic methods, e.g. ³¹P NMR.

Due to the particular combination of strength (in particular wet-strength) and water absorption, the spun mat or filaments described herein may be used as a water-absorbent material. A hygiene product is therefore provided which comprises the spun mat or filaments of the invention and/or a water-absorbent material comprising said spun mat or filaments. The hygiene product may be selected from the group consisting of a disposable diaper, a sanitary napkin, a wipe, a tampon, an absorbent dressing and a disposable tissue. A method for providing a hygiene product is also provided, said method comprising preparing a mat or filaments of crosslinked phosphorylated microfibrillated cellulose according to the invention, and; incorporating said mat or filaments into a hygiene product. The skilled person is aware of standard methods for constructing hygiene products, and incorporating mats or filaments into such products.

### EXAMPLES

1. Dry-spinning of phosphorylated microfibrillated cellulose (P-MFC)

Materials:
1. P-MFC 1: DS=0.63 mmol/g; pH=9.5; ˜1.5 wt %
2. P-MFC 2; DS=0.86 mmol/g; pH=9.3; ˜1.5 wt %

Experimental:

P-MFC was spun directly onto aluminum foil using a 20 mL plastic syringe without needle. Single filaments, grid and random mat patterns were created. The spun materials were placed in the oven at 105° C. for 40 min in order to dry.

The aluminum foils with dry spun P-MFC samples were soaked in deionized water for about 2 hours. After soaking the spun material swelled and became easier to separate from the aluminum foil.

Observations:

The re-wetted materials presented a rather flexible character and some stretchability/elasticity, as assessed manually. The swelling capacity of both grades of P-MFC upon heat-treatment at different temperatures and subsequent soaking in water were tested:

P-MFC 1 (70° C.)=19.18±1.76 g water/g

P-MFC 1 (105° C.)=10.80±0.26 g water/g

P-MFC 2 (70° C.)=20.40±1.32 g water/g

P-MFC 2 (105° C.)=14.19±2.36 g water/g

From these results, P-MFC with higher degree of modification, meaning more negative charges (P-MFC 2), can swell more. Also, the temperature of heat-treatment influences the swelling capacity (higher temperature=less swelling), meaning that, in principle, it also affects the crosslinking extent (more crosslinking at higher temperature).
2. Dry-spinning of P-MFC and other charged and native MFC grades

Effect of different MFC grades and drying conditions (comparative examples)

Materials:
1. P-MFC 2: DS=0.86 mmol/g; pH=9.3; ˜1.5 wt %
2. Native MFC: pH=6.5; ˜4.5 wt %
3. TEMPO-MFC: DS=1.25 mmol/g; pH=10.5; ˜1.94 wt %
4. Cationic MFC: DS=1.0 mmol/g; pH=10.6; ˜1.5 wt %

(Note: Native MFC had much lower viscosity than the charged grades, so a higher solids content was used for spinning.)

Experimental:

Various MFCs were spun directly onto aluminium foil using a 20 mL plastic syringe without needle. Single filaments, grid and random mat patterns were created.

The spun materials were put in the oven at 105° C. for 40 min in order to dry, except for the spun P-MFC samples, which were left to dry at ambient conditions (approximately 25° C.).

The aluminum foils with dry spun materials were soaked in deionized water for about 2 hours.

Observations:
TEMPO-MFC formed thick threads when extruded.
P-MFC was the easiest grade to extrude.
Cationic MFC was the second easiest grade to extrude.
After drying native MFC, a very brittle material was obtained, and great part of the grid pattern broke during drying. When submerged in water the samples detached from aluminum foil, but samples were very fragile and hard to handle. No stretchability or flexibility behavior was observed.
TEMPO-MFC formed very flat samples and did not swell significantly after soaking in water. No stretchability was observed.
Cationic MFC swelled very much upon soaking in water, but the “gelified” samples were very fragile and hard to handle and separate from aluminum foil. No stretchability was observed.
P-MFC dried at ambient conditions got very swollen upon soaking in water, but samples were very fragile and hard to handle. Also no stretchability was noted.

Main conclusions:
Heat treatment is needed to attain stretchable/elastic behaviour.
Stretchable/elastic behaviour is particular to phosphorylated MFC.
The elastic behaviour is an indication of crosslinking.
3. Effect of spinning P-MFC together with native (unmodified) MFC Mixtures of P-MFC and native MFC at different weight ratios, namely 25:75, 50:50 and 75:25 P-MFC/MFC ratio were formulated.

It was observed that all mixtures could form filaments and spun mats. The extreme shrinkage and brittleness typical of native MFC was not observed. Also the samples with only 25% of P-MFC swelled very little in water and did not show signs of elasticity in wet-state. Samples with 50% of P-MFC swelled more than those with only 25%, but already presented some signs of elasticity in wet-state. Samples with 75% of P-MFC provided the highest swelling capacity and elasticity in wet-state.

1. A method for preparing a fibrous material of crosslinked microfibrillated cellulose, said method comprising the steps of:
i. spinning a cellulose composition comprising phosphorylated microfibrillated cellulose (P-MFC) into a fibrous material; and
ii. heat-treatment of said fibrous material to provide crosslinking of the phosphorylated microfibrillated cellulose.

2. The method according to claim 1, wherein said fibrous material is filaments.

3. The method according to claim 1, wherein said fibrous material is a mat.

4. The method according to claim 1, wherein said cellulose composition further comprises unmodified microfibrillated cellulose.

5. The method according to claim 1, wherein said cellulose composition further comprises chemically-modified microfibrillated cellulose.

6. The method according to claim 1, wherein said cellulose composition comprises more than 25% by weight P-MFC.

7. The method according to claim 1, wherein said heat treatment takes place at a temperature of between 60 and 200° C.

8. The method according to claim 1, wherein said heat treatment takes place for a time of between 10 and 180 minutes.

9. The method according to claim 1, wherein the crosslinking takes place in the absence of any additional crosslinking agents.

10. The method according to claim 1, wherein the spinning is selected from the group consisting of wet-spinning, electrospinning and dry-spinning.

11. The method according to claim 1, wherein said phosphorylated microfibrillated cellulose (P-MFC) is obtained by;
providing a suspension of cellulose pulp fibers in water, and
phosphorylating the cellulose pulp fibers in said water suspension with a phosphorylating agent, followed by fibrillation.

12. The method according to claim 1, further comprising the step of drying said fibrous material, before the heat-treatment step.

13. The method according to claim 1, further comprising the step of hydrating said fibrous material with water after the heat-treatment step.

14. A spun mat or spun filaments obtained via the method of claim 1.

15. A spun mat or spun filaments comprising crosslinked phosphorylated microfibrillated cellulose.

16. A method for preparing a web of spun filaments, said method comprising:
preparing spun filaments of crosslinked phosphorylated microfibrillated cellulose according to claim 1, and;
laying said spun filaments to provide a web.

17. A web comprising spun filaments, wherein said spun filaments are according to claim 14.

18. The web according to claim 17, or method according to claim 16, wherein said web further comprises additional filaments or fibres.

19. The web according to claim 17, wherein said web is woven or non-woven.

20. A water-absorbent material comprising the mat or filaments according to claim 14.

21. A hygiene product comprising the mat or filaments according to claim 14.

22. The hygiene product according to claim 21, wherein the hygiene product is selected from the group consisting of a disposable diaper, a sanitary napkin, a wipe, a tampon, an absorbent dressing and a disposable tissue.

23. A method for providing a hygiene product, said method comprising:
preparing a fibrous material of crosslinked microfibrillated cellulose according to claim 1, and;
incorporating said mat or filaments into a hygiene product.
